(19) United States
(12) Patent Application Publication
(10) Pub. No.: US 2014/0357976 A1
(43) Pub. Date: Dec. 4, 2014

(54) MENTAL STATE ANALYSIS USING AN APPLICATION PROGRAMMING INTERFACE

(71) Applicant: Affectiva, Inc., Waltham, MA (US)

(72) Inventors: Boisy G. Pitre, Opelousas, LA (US); Rana el Kaliouby, Boston, MA (US); Youssef Kashef, Obour City (EG)

(21) Appl. No.: 14/460,915

(22) Filed: Aug. 15, 2014

Related U.S. Application Data
(63) Continuation-in-part of application No. 13/153,745, filed on Jun. 6, 2011.
(60) Provisional application No. 61/867,007, filed on Aug. 16, 2013.
(60) Provisional application No. 61/924,252, filed on Jan. 7, 2014.
(60) Provisional application No. 61/916,190, filed on Dec. 14, 2013.
(60) Provisional application No. 61/927,481, filed on Jan. 15, 2014.
(60) Provisional application No. 61/953,878, filed on Mar. 16, 2014.
(60) Provisional application No. 61/972,314, filed on Mar. 30, 2014.
(60) Provisional application No. 62/023,800, filed on Jul. 11, 2014.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.

(51) Int. Cl. A61B 5/16; A61B 5/00

(52) U.S. Cl. CPC A61B 5/165; A61B 5/0077; A61B 5/6898
USPC 600/407

(57) ABSTRACT

A mobile device is emotionally enabled using an application programming interface (API) in order to infer a user's emotions and make the emotions available for sharing. Images of an individual or individuals are captured and send through the API. The images are evaluated to determine the individual's mental state. Mental state analysis is output to an app running on the device on which the API resides for further sharing, analysis, or transmission. A software development kit (SDK) can be used to generate the API or to otherwise facilitate the emotional enablement of a mobile device and the apps that run on the device.

### RELATED APPLICATIONS

This application claims the benefit of U.S. provisional patent applications “Application Programming Interface for Mental State Analysis” Ser. No. 61/867,007, filed Aug. 16, 2013, “Mental State Analysis Using an Application Programming Interface” Ser. No. 61/924,252, filed Jan. 7, 2014, “Heart Rate Variability Evaluation for Mental State Analysis” Ser. No. 61/916,190, filed Dec. 14, 2013, “Mental State Analysis for Norm Generation” Ser. No. 61/927,481, filed Jan. 15, 2014, “Expression Analysis in Response to Mental State Express Request” Ser. No. 61/953,878, filed Mar. 16, 2014, “Background Analysis of Mental State Expressions” Ser. No. 61/972,314, filed Mar. 30, 2014, and “Mental State Event Definition Generation” Ser. No. 62/023,800, filed Jul. 11, 2014. This application is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011. The foregoing applications are each hereby incorporated by reference in their entirety.

### FIELD OF ART

This application relates generally to application programming interfaces and software development kits and more particularly to an application-programming interface for mental state analysis.

### BACKGROUND

The wide varieties and sizes of computer systems that are available today require software developed according to a wide variety of standards. Modern software is highly complex and includes a wide range of capabilities, features, and user selectable options. Software systems often contain hundreds of thousands to millions of lines of code or more. As a result, software development has become a major worldwide industry. This industry continues to expand, resulting in a host of companies continuously developing new products, improving feature sets, and generally updating their respective software products. The types of software developed are often described by broad categories that are based on the intended uses of the software. The software is categorized by type, for example: systems software, including operating systems; utility software for monitoring and configuring computer systems; device drivers for attached peripherals such as disks and displays; graphical user interfaces for improving the human-computer interface, etc.; compilers for assembly, procedural, scripting, and object-orientated programming languages; and application software, to name only a few. Each software type covers a broad range of potential software uses within a specific category. For example, application software covers such diverse purposes as enterprise software, security software, and industry-specific software. Enterprise software includes products for asset management and customer relationship management, in addition to purpose-built codes for financial institutions, travel agencies and booking agents, telecommunications companies, healthcare applications, retail stores, and others. Due in part to the complexity and development costs of these and other software systems, software developers must produce their codes cost-effectively and efficiently.

Software is developed using a wide range of engineering and productivity models. Software developers can choose to build their software from a conglomeration of code, often choosing to obtain code modules from a variety of sources and vendors rather than developing their codes entirely in-house. And, in turn, the code developed by an organization is often offered to and purchased by both other software developers and end users or customers. Obtaining previously written code modules can simplify the process of code development across many sectors. For example, a developer of code for a medical application can choose to concentrate on effectively and efficiently coding the complexities of his or her image processing algorithm and opt to purchase code to handle other tasks such as data acquisition and storage, wireless sensing and communication, graphical user interfaces, etc. Thus, such modular code and the completed code containing modular elements must function properly, be stable and reliable, and be easily maintained. The software modules sold by software producers are meticulously designed so that purchasers of the software are able to easily integrate the modules into their products and systems. The purchasers, in turn, often sell their software products and services on to other purchasers who continue the integration process. The software interface controlling the entire integration and implementation of the modular code elements is called the application-programming interface (API). The API describes how various pieces of software, or software components, are expected to work together. The API specifies software interactions, use of databases, etc., and gives information on how to access various computer hardware components such as hard disk drives (HDD), solid state disk drives (SSD), input/output (I/O) devices, graphics devices, and so on. The API defines how to interact with software modules such as graphics packages, equation solvers, etc. The API is often a library which includes specifications for all computational routines, access to and use of data structures and variables, and in the case of object oriented programming, various object classes. The API provides the clearly defined programming interfaces so that the development effort is eased.

### SUMMARY

Portable devices are “emotionally enabled” by an application programming interface (API) to determine the mental state of a user or users. Images are gathered of the user and analyzed using one or more classifiers. The classifiers are downloaded from a website to the portable device. The number and type of classifiers downloaded depend on the processing capability, communications bandwidth, and storage capabilities of the device. A computer-implemented method for application programming interface usage is disclosed comprising: obtaining images of an individual through a software interface on a device; evaluating the images to determine mental state data; analyzing mental state information based on the mental state data; and outputting the mental state information through the software interface. The software interface can be considered an application programming interface (API). The application programming interface can be generated by a software development kit (SDK).

In embodiments, a computer program product embodied in a non-transitory computer readable medium for mental state analysis comprises: code for obtaining images of an individual through a software interface on a device; code for evaluating the images to determine mental state data; code for analyzing mental state information based on the mental state data; and code for outputting the mental state information through the software interface. In some embodiments, a computer system for mental state analysis comprises: a memory which stores instructions; one or more processors attached to the memory wherein the one or more processors, when executing the instructions which are stored, are configured to: obtain images of an individual through a software interface on a device; evaluate the images to determine mental state data; analyze mental state information based on the mental state data; and output the mental state information through the software interface.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for an API for mental state analysis.

FIG. 2 is an example API with classifiers.

FIG. 3 is an example diagram of apps calling the API.

FIG. 4 is an example diagram showing collection of mental state data from multiple devices.

FIG. 5 is an example system that uses an API for mental state analysis.

FIG. 6 is a system for API generation.

### DETAILED DESCRIPTION

Assessment of individual emotional reactions to life events, media presentations, and communication with other people, to name a few, has become ubiquitous in modern, social-media saturated society. From the moment a person wakes to the moment they go to bed, the opportunities for assessing, self-quantifying, and sharing emotional reactions to even the most mundane of daily activities are abundant. For example, social media sites such as Facebook™, Digg™, and Google+™, among others, allow a person to instantly and broadly share his or her emotional reaction to a meal at a restaurant, the view from the upper floor of a building, his or her partner returning from a trip, among numerous other situations. With the ubiquitous nature of smartphones and web-connected devices such as tablets, cameras, and even watches and other ultra-portable wearable devices, the opportunities to share emotional reactions are staggering.

Yet, even with the numerous opportunities to share mental state information, perhaps the most significant barrier to a person's ability to share his or her mental state remains the difficulty and time-consuming nature of self-accessing emotional reactions to life events, media, and other emotionally charged situations. Disclosed below are concepts which emotionally enable a device to employ various clarifiers from within the framework of an application programming interface (API) to provide a reliable way to evaluate and communicate an accurate representation of the user's mental state. Applications can call the API, send information to the API (including video or images), and receive mental state analysis sent back from the API. In embodiments, the applications are referred to as apps.

The disclosed API for emotionally enabling devices allows for unconscious analysis of facial expressions, body language, and other corporeal reactions in much the same way a friend might analyze a person's mental state quickly, and with a minimum of conscious effort. Using advanced classifiers in concert with the cameras or other imaging devices present in the vast majority of internet-connected personal electronic devices such as smartphones and tablets, the disclosed API allows images of a person to be effectively analyzed and rendered as pertinent, sharable information. For example, the API can work in concert with a smartphone operating system to employ images or videos obtained from a front-facing camera of a user's smartphone to analyze the person's emotional state while watching or after finishing a YouTube™ video or another media presentation. The smartphone could use the disclosed API in combination with various applications in order to obtain images and then evaluate a user's mental state. The user's mental state can then be used by the app to evaluate different aspects of the user's mental response based on the app's intended function. If it is determined that the user had a negative emotional reaction to the media, the user can be presented with a dialogue asking whether the user wants to share his or her reaction with other people. For example, the sharing can comprise a pre-composed image containing an image of the user at the height of his or her emotional response placed beside or above an image of a specific point in the media and captioned: “‘User X’ did not enjoy watching ‘video title.’” In this manner, the user is presented with a convenient and accurate way to share his or her response to media. In the same manner, the user's smartphone or tablet camera can capture images of the user as the user performs daily tasks such as checking email, online banking, and logging exercise or daily food consumption. Using the API and classifiers, an app on the system can analyze such images and determine at what point during the day the user had the most positive emotional response. The app could then present the user with a dialogue first asking “Were you browsing ‘x’ website at 2:34 p.m.?” If the user answers in the affirmative, another dialogue might ask “Would you like to share the following image on a social media site?” accompanied by a pre-composed image of the user at the height of his or her emotional response and a caption such as, “‘X user’ was happiest today when ‘X user’ was browsing ‘Y website’ at 2:34 p.m.” The app can also listen for a specific emotional event, and when the event is detected, use the API to perform analysis on images in order to create usable mental state information.

Thus, a user's personal electronic device can be emotionally enabled, with the API allowing for both the efficient transfer of mental state information between applications and the effective analysis of images. Apps or other user interfaces on the device can then use the mental state information acquired through the transfer to conveniently present individuals with various opportunities to fluidly and intuitively share and understand personal moods, emotions, and emotional states. As an effect of the ability to present the user with his or her own emotional states, the user avoids the cumbersome and often overwhelming task of subjectively analyzing and vocalizing emotional states and moods.

To facilitate the generation of mental state data, images of one or more individuals whose mental states are sought are collected. In embodiments, images of people posting to social networks who desire to share their mental states with other social network users can be collected. The images are analyzed using one or more classifiers that are obtained from a web service. The image capture can be performed using a variety of imaging devices, including cameras on portable devices, cameras on stationary devices, and standalone cameras, provided that the imaging devices are accessible through a software interface on the emotionally enabled personal electronic device or devices. Additionally, the interface also allows some or all processing of the images to be performed locally on the device instead of sending the images for analysis, depending on the processing, storage, and communication capabilities of the device.

Having obtained one or more images of an individual through a software interface on a device, an evaluation of the images is performed. Similarly, one or more images can be obtained from a plurality of people using software interfaces on one or more devices. The image evaluations provide insight into the mental states of the users. All or part of the image evaluation can take place on a portable device. Through evaluation, many different mental states can be determined, including frustration, confusion, disappointment, hesitation, cognitive overload, focusing, engagement, attention, boredom, exploration, confidence, trust, delight, disgust, skepticism, doubt, satisfaction, excitement, laughter, calmness, stress, and curiosity. Other mental states can be determined through similar evaluation.

Mental state information of an individual or plurality of individuals is output through the API on the device. The output can include text, figures, images, video, and other data relating to the mental state or states of the individuals whose images were analyzed. The mental states can be rendered on a social network for sharing with other users of the social network. A user posting to a message or image to a social network can choose to include additional information with the posting including the rendering of his or her mental state or states. The posting of a representation of an analyzed mental state of the user provides viewers of the post with a keen insight into the mental state of the user at the time of the posting.

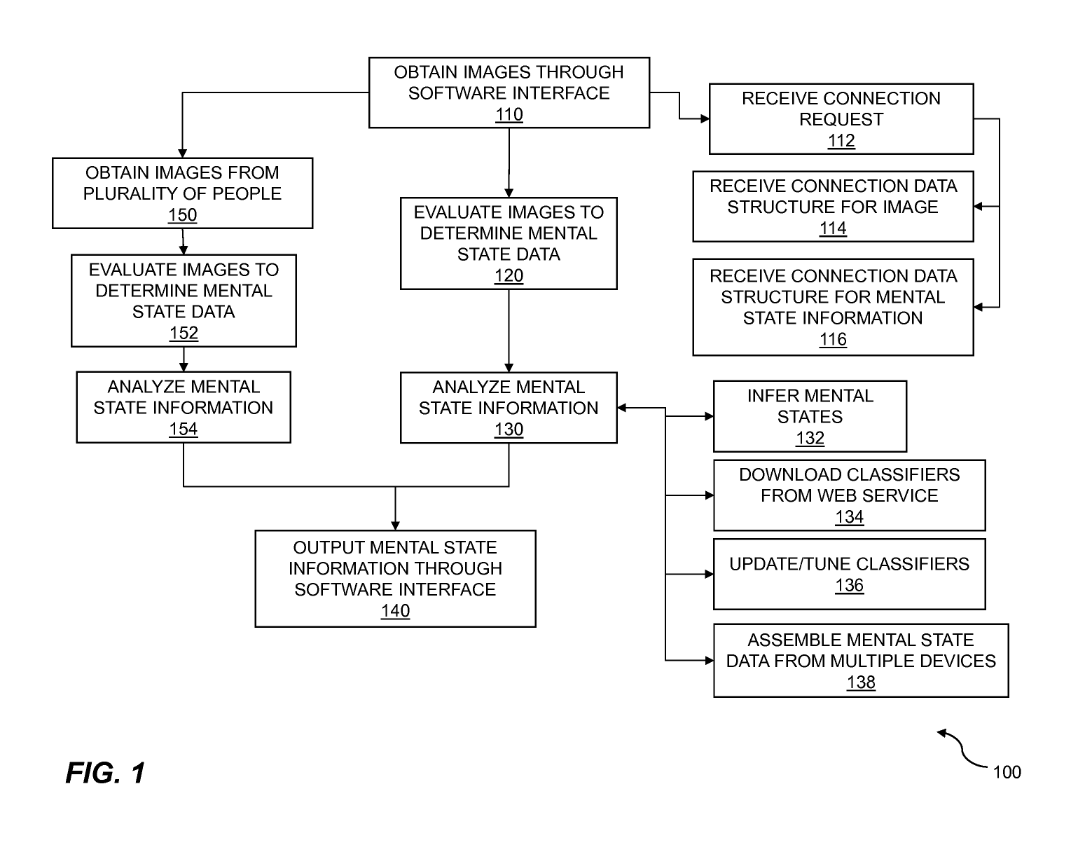

FIG. 1 is a flow diagram for an API for mental state analysis. A flow 100 that describes a computer-implemented method for application programming interface usage is shown. The flow 100 includes obtaining images of an individual through a software interface on a device 110. The device can be a mobile device or any other device suitable for image capture. The obtaining through the software interface can be accomplished by accessing software on a device. In embodiments, the images are collected from a video of the individual. The images can be collected to evaluate mental state information. The device can be a portable device equipped with a camera. The device can be a smartphone, PDA, tablet, laptop, or any other portable device. The camera can be built into the device, connected to the device wirelessly or with a tether, and so on. Image data can be captured by using any type of image-capture device including a webcam, a video camera, a still camera, a thermal imager, a CCD device, a phone camera, a three-dimensional camera, a light field camera, multiple cameras to obtain different aspects or views of a person, or any other type of image capture technique that allows captured data to be used in an electronic system. The software interface can be an app on the portable device. The app can be one or more of many apps on a portable device, where the apps can be installed by default on the portable device, can be installed by the user of the device, and so on. The software interface includes an application programming interface (API). The API can permit both developers and users alike to access the device and to develop apps for the device. Access to the device can allow for running of programs, setting of default values, controlling attached devices, gathering data from attached devices such as cameras, and so on. In embodiments, the API is generated by a software development kit (SDK). The SDK can be executed on the portable device or can be executed on a separate computer system such as a tablet, a laptop, a desktop, a workstation, a mainframe, or any other appropriate computer system. In embodiments, the SDK runs on a different system from the device. The SDK can be used to generate any type of app for the portable device. In embodiments, the software development kit is specifically designed to develop apps for the device to be emotionally enabled. In other embodiments, the SDK is for a family of devices, such as a class of tablet or smartphone running a certain operating system; multiple devices, such as a SDK able to develop cross-platform apps; and so on.

The flow 100 continues by receiving a connection request 112 through the software interface for the images and the mental state information. The request can originate from an action initiated by the user such as starting an app, scheduling an app, pressing a button on a device display, pressing a physical button, and so on. The request can come from a system, an application, or programming running on a remote processor. For example, a request for an image and mental state can come from a social networking site. In embodiments, the connection request comes from a peer component, i.e. a component accessed independently of an external network connection. A peer component can be a hardware component or a software component. The peer component can make a request as a result of an action taken by a user or can result from a request originating externally to the device. In embodiments, the peer component is also running on the same device as the API. The peer component can be a component of the operating system on the device, or can be added by a user or by another application.

The flow 100 continues with receiving a connection data structure 114 for the connection request where the connection data structure describes a format for the images. To facilitate image transmittal and processing, an app requesting an image must know where and in what format the image is stored. The data connection structure can allow an app to define an access route to an image. The accessing of the image can take any of a number of paths, including direct access to a camera or other image capture device, access through an API, or access through another type of data structure. The data structure can include an appropriate number of fields necessary to describe the image. The data structure can also provide key information about the potentially accessed image, including number of bytes in the image, byte order, image encoding format such as TIFF or JPEG, image name, GPS coordinates, and so on. Following the same access pattern, the flow 100 also includes receiving a connection data structure for the connection request where the connection data structure describes a format for the mental state information 116. As was the case for the image, a data structure can provide a path by which an app or another system request can access and use mental state information. Ready access to mental state information, along with format data for the mental state information, means that using a data structure can greatly simplify the process of integrating mental state information into the function of an app or device.

The flow 100 includes evaluating the images to determine mental state data 120. The evaluation of the images can include various image-processing techniques. The evaluating can including searching the images for key facial features or pointers that can be used to determine mental state data. The mental state data can include facial expressions, general facial expressiveness, changes in facial expression, and so on. Facial pointers can include a smile, a frown, a measure of eyebrow raises, and so on. In some embodiments, one or more apps run in the background and images continue to be evaluated for mental state analysis even when the apps are not being displayed or are not running in the foreground of a device. In embodiments, the mental state data can be determined as a result of a user initiating an app, an app running on a schedule, a button press, and so on. In embodiments, the mental state data is collected sporadically. The collection of mental state data can be dependent on the location of the user's device, time of day, day of week, and so on. The sporadic collection can be due to the bandwidth capability of the device, changes in or intermittent connectivity, and so on.

The flow 100 includes analyzing mental state information 130 based on the mental state data. The mental state data that was collected as a result of one-time collection, periodic collection, sporadic collection, and so on, can be analyzed to produce mental state information. The resulting mental state information can be analyzed to infer mental states 132. The mental states include one or more of stress, sadness, happiness, anger, frustration, confusion, disappointment, hesitation, cognitive overload, focusing, engagement, attention, boredom, exploration, confidence, trust, delight, disgust, skepticism, doubt, satisfaction, excitement, laughter, calmness, and curiosity, among other mental states. A variety of analysis techniques can be used. In embodiments, the software interface makes available one or more analyzing functions for the mental state data. Any number of analyzing functions appropriate for analysis of mental state data can be used.

The analyzing is based on one or more classifiers, which can process mental state data based on conformity to certain criteria in order to produce mental state information. Any number of classifiers appropriate to the analysis can be used. The classifiers can be obtained by a variety of techniques. The flow 100 continues with downloading the one or more classifiers from a web service 134, but the classifiers can also be obtained by a variety of other techniques including an ftp site, a social networking page, and so on. In embodiments, the one or more classifiers for downloading are based on one or more of processing capability of the device, bandwidth capability for the device, and memory amount on the device, allowing classifiers appropriate to the capabilities of the device to be downloaded. For example, if the device has a relatively low computational capability, simple classifiers can be downloaded to start the processing.

The flow 100 further comprises updating the one or more classifiers 136 or in some cases tuning the one or more classifiers 136 based on improvements to the one or more classifiers in cloud storage. The classifiers can be tuned or updated based on a number of factors, including real-time performance evaluation obtained from the device. By updating or tuning the classifiers, improved accuracy of mental state analysis can be achieved without updating the app or the API. Further, there is no need to compile or relink any of the program code.

In certain embodiments, it is useful to assemble mental state data from multiple devices 138 and use the assembled mental state data in the analyzing. Assembling mental state data from multiple devices can provide a more thorough view of an individual's or group of individuals' mental states than would be possible using only one source of mental state data. For example, building a timeline of mental state data by assembling mental state data from multiple devices can create a more complete set of mental state data across the timeline. At points along the timeline, mental state data might be available from multiple sources. At those points the best or most useful mental state data can be selected. The utility of mental state data can be determined by various factors, including assigning the highest rank to data from the camera with the most direct view of the individual or by using other criteria. In some cases the mental state data can be combined, such as by averaging the mental state data. In other cases the multiple pieces of mental state data can be retained so that later mental state analysis can use each of the pieces of data. Thus, assembling mental state data from multiple devices can improve mental state data coverage, aid in ensuring the accuracy of mental state data, and improve long-term usefulness of a particular set of data by allowing a more nuanced re-evaluation of the data at a later time.

The flow 100 also includes outputting the mental state information through the software interface 140. The mental state information can include a connection data structure that describes a format for the images, and a connection data structure that describes the format for the mental state information. The mental state information can be used to infer one or more mental states of the user whose image is obtained. The mental state information which is output can be used to determine smiles, project sales, determine expressiveness, analyze media, monitor wellbeing, evaluate blink rate, evaluate heart rate, evaluate humor, and post to a social network. The outputting can include displaying images, text, emoticons, special icons, special images, and any other output format able to represent the one or more mental states. The posting of mental state can include posting to one or more social networks. The social networks can include social networks to which the user is subscribed. Further, mental states can be inferred for a plurality of people. When mental states can be inferred for a plurality of people, the plurality of people can share the mental states on social networks. The plurality of people can be in one or more social networks. The mental states can be shared with all members of a social network, some members of a social network, a specific group of members of a social network, and so on.

Additionally, the flow 100 can include obtaining images for a plurality of people 150. The images of the plurality of people can be used to determine mental state analysis of an individual within the plurality of people. Facial analysis can be used to identify the individual within the plurality of people. Additionally, mental state data captured on the individual can be correlated with mental state data captured on both the individual and plurality of other people simultaneously to aid in inferring mental states for the individual. As is the case for the single user described above, one or more devices can be used for the capture of the images. The devices can include one or more mobile devices or any other device or devices suitable for image capture. The images can be obtained through a software interface. The obtaining through the software interface can be accomplished by accessing software on a device. The images can be collected from one or more videos of the plurality of users. The one or more devices can be portable devices equipped with cameras. The devices can be smartphones, PDAs, tablets, laptops, or any other portable devices. The one or more cameras can be built into the one or more devices, connected to the devices wirelessly or with tethers, and so on. Image data can be captured by using any type of image-capture device or combination of devices including a webcam, a video camera, a still camera, a thermal imager, a CCD device, a phone camera, a three-dimensional camera, a light field camera, multiple cameras to obtain different aspects or views of a plurality of people, or any other type of image capture technique that can allow captured data to be used in an electronic system.

In other embodiments, images can be obtained for a plurality of people 150, so that evaluating can be performed on the images for the plurality of people to determine mental state data 152 for the plurality of people. As with evaluating images from a single individual, key facial features or pointers that can be used to determine mental state data can be extracted for the plurality of people. The mental state data can include facial expression, general facial expressiveness, changes in facial expression, and so on. Facial pointers can include a smile, a frown, eyebrow raises, and so on. As was the case for mental states inferred from an individual, the mental state information for the plurality of people can be output through the software interface.

The flow 100 continues with analyzing mental state information 154 for the plurality of people based on the mental state data for the plurality of people in order to infer one or more mental states. The mental states can include one or more of frustration, confusion, disappointment, hesitation, cognitive overload, focusing, engagement, attention, boredom, exploration, confidence, trust, delight, disgust, skepticism, doubt, satisfaction, excitement, laughter, calmness, stress, and curiosity. As was the case for the analysis of mental state data for a single user, a variety of analysis techniques can be used, including analysis performed using a software interface or another appropriate analysis method. The analyzing can be based on one or more classifiers that can be downloaded from a web service. As with mental state data for an individual, mental state data from multiple devices can be assembled to aid in analyzing mental state data for the plurality of people. The devices which can produce mental state data on the plurality of people include one or more of a webcam, a video camera, a still camera, a thermal imager, a CCD, a phone camera, a three-dimensional camera, a light field camera, multiple cameras or sensors, or any other type of image capture technique that can allow captured data to be used in an electronic system.

The flow 100 continues with outputting the mental state information for the plurality of people through the software interface 140. As was the case with the individual, outputting the mental state information can take place using any software interface, such as a social network to which the plurality of people are subscribed. Additionally, the outputting can take place on any interface capable of receiving and displaying the mental state information. Various steps in the flow 100 may be changed in order, repeated, omitted, or the like without departing from the disclosed concepts. Various embodiments of the flow 100 may be included in a computer program product embodied in a non-transitory computer readable medium that includes code executable by one or more processors.

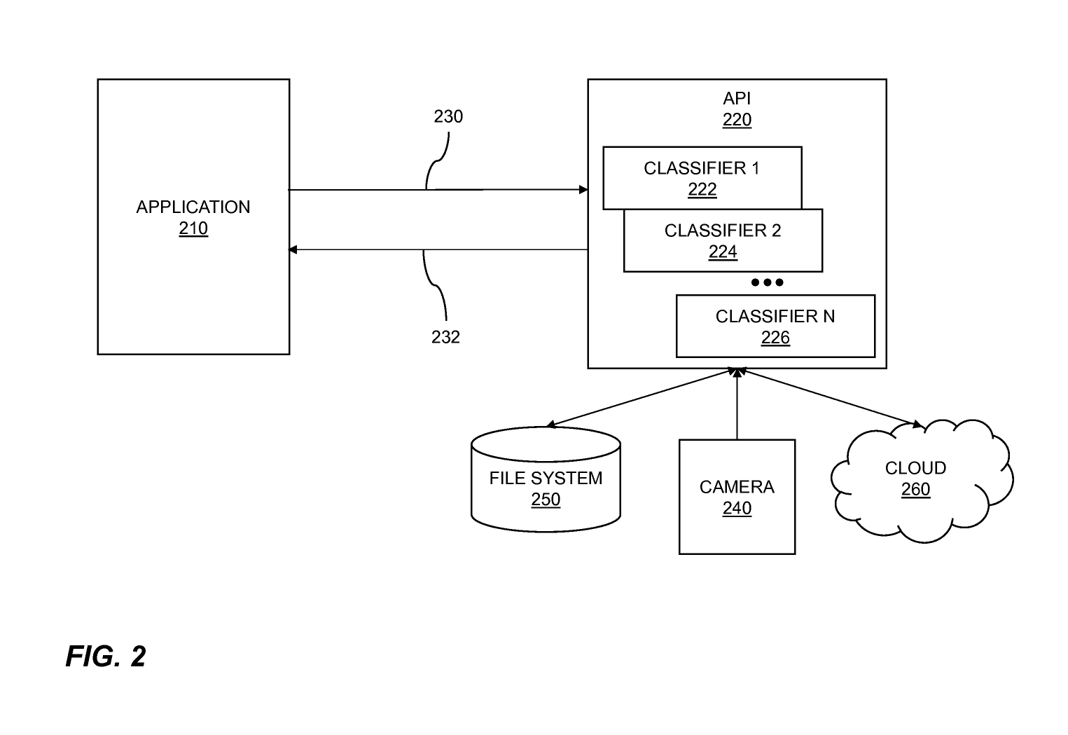

FIG. 2 is a diagram of an example API using classifiers. An application 210, or app, is shown loaded onto a device. Any number of apps can be loaded or running on the device. The app can be a social networking app, such as Facebook™, Digg™, Google+™, LinkedIn™, Tumblr™, and so on. Numerous other types of apps can likewise utilize emotional enablement. Emotional enablement of an app can allow a user to automatically express his or her emotions while using the app. The device running the app can be any of a range of devices including portable devices such as laptop computers and ultra-mobile PCs, mobile devices such as tablets and smart phones, wearable devices such as glasses and wristwatches, and so on. In many cases, the devices contain built-in cameras, but some devices might employ external cameras connected to the device, accessible by the device, and so on.

In the example shown, an application 210 communicates through an API 220 which allows for emotionally enabling the application. In some embodiments, the API 220 is generated by a software development kit or SDK. The API 220 shown includes multiple classifiers to process mental state data and infer mental states. The mental states can include one or more of stress, sadness, anger, happiness, frustration, confusion, disappointment, hesitation, cognitive overload, focusing, engagement, attention, boredom, exploration, confidence, trust, delight, disgust, skepticism, doubt, satisfaction, excitement, laughter, calmness, and curiosity. One or more mental states can be analyzed to determine emotional states, moods, and other useful information which can prove difficult for an individual to self-identify. In embodiments, one or more classifiers are present in an API. In the figure, three example classifiers are shown, classifier 1 222, classifier 2 224, and classifier N 226. While classifiers are typically code or data from a cloud or other source, in some cases classifiers can be stored locally on the device. In embodiments any number of classifiers are possible. The classifiers can be obtained from any of a variety of sources including by Internet download, from an application vendor site, from user-developed code, and so on. Similarly, new classifiers can be obtained from a variety of sources. The classifiers in the API can be updated automatically.

Various communication channels can exist between an application and an API. For example, the application 210 can communicate with the API 220 via channel 230, and can receive a communication back from the API via the same channel or another channel, such as second channel 232. The API 220 can receive an initialization or another communication 230 from an application 210. The application can use delegation techniques for operations performed by the API and can include delegating a message from the API to an application. Delegation is a design pattern in software by which one entity relays a message to another entity, or performs an action based on the consent of the entity. An entity can be considered an instantiated object of a class within object oriented environments. The API can perform various operations based on the initialization. The operations performed can include one of more of the classifiers 1 through N. Information on one or more emotional states can be returned using a channel 232 to the application 210.

The API 220 uses classifiers to process and analyze mental state data gathered from a user or users. In embodiments, the data is in the form of an image or video of the user or users. The image or video can be obtained from a variety of sources including one or more cameras 240, file systems 250, or cloud-based resources 260, and can be obtained using a variety of networking techniques including wired and wireless networking techniques. In embodiments, the images can be from a collection of photographs, an album, or other grouping of images or videos. The application can pass parameters or information on the source of the video or images that contains mental state data to the API. Mental state information, when analyzed from the mental state data, can aid individuals in identifying emotional states and moods. In embodiments, the application, API, camera 240, and file system 250 reside on the same device.

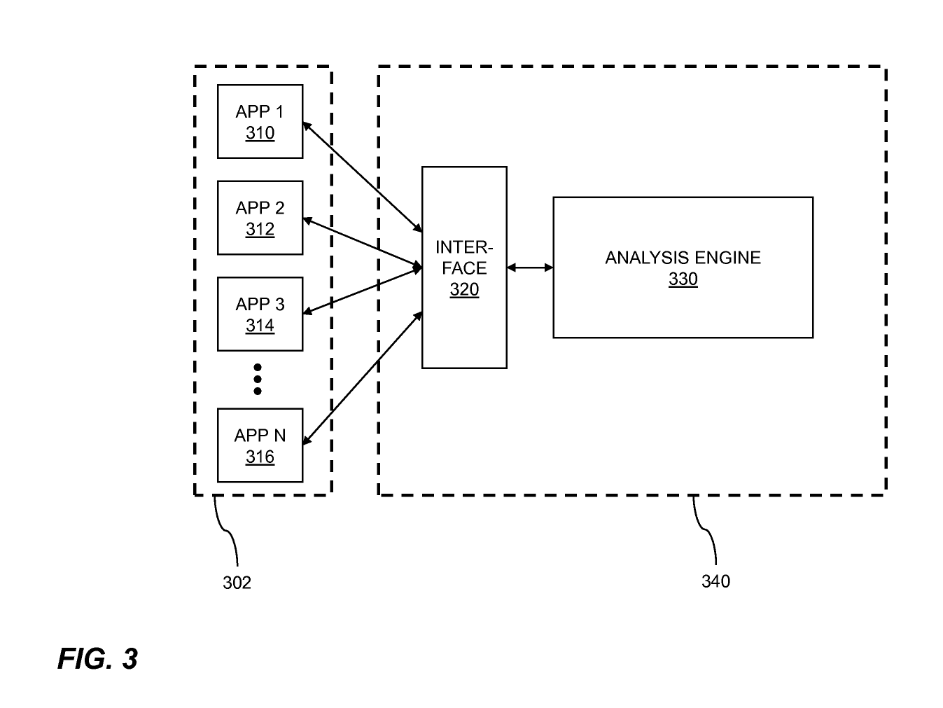

FIG. 3 is an example diagram of apps calling the API. In the example, one or more apps 302 call an API 340. The apps can reside on a device, where the device can be a portable device such as a laptop or ultra-mobile PC, a mobile device such as a smartphone or tablet, a wearable device such as glasses or a watch, and so on. In embodiments, the apps 302 and the API 340 can reside on the same device. The apps 302 can include a single app such as app 1 310. In some embodiments, the apps 302 comprise a plurality of applications, such as app 1 310, app 2 312, app 3 314, app N 316, and so on. The apps can comprise any of a variety of apps including social media apps. The API 340 provides emotional enablement to a device on which the API 340 resides. The user can choose to emotionally enable any number of apps loaded on her or his device. The one or more apps 302 can send video, images, raw data, or other user information to the API 340 for analysis. The images, video, user information, and the like, can be generated by the device, obtained by the device, loaded onto the device, and so on.

The API 340 can include an interface 320 for receiving raw data and for sending mental state data, mental state information, and emotional state information to and from the one or more apps 302. The raw data received by the interface 320 can include images, video, user information, etc. The interface can provide a variety of functions to assist communication between the apps and the analysis engine. The interface 320 can communicate with the one or more apps using any of a variety of techniques including wired networking using USB, Ethernet, etc., wireless networking using Wi-Fi, Bluetooth®, IR, etc., or another appropriate communication technique. The communications between the one or more apps and the interface can be unidirectional or bidirectional.

The API 340 includes analysis capabilities in the form of an analysis engine 330. In some embodiments, the API 340 also communicates with a web service. Analysis of the raw data can be performed on the device, on the web service, or on both. An app on a device can use delegation for analysis of data by the API. The raw data can include images, video, user information, and so on. In at least one embodiment, all of the analysis needed by the one or more apps 302 is performed on the device. The analysis engine 330 can analyze the image or video to determine one or more mental states, where the mental states can include one or more of stress, sadness, happiness, anger, frustration, confusion, disappointment, hesitation, cognitive overload, focusing, engagement, attention, boredom, exploration, confidence, trust, delight, disgust, skepticism, doubt, satisfaction, excitement, laughter, calmness, and curiosity. The analysis engine can determine one or more emotional states based on the mental state information.

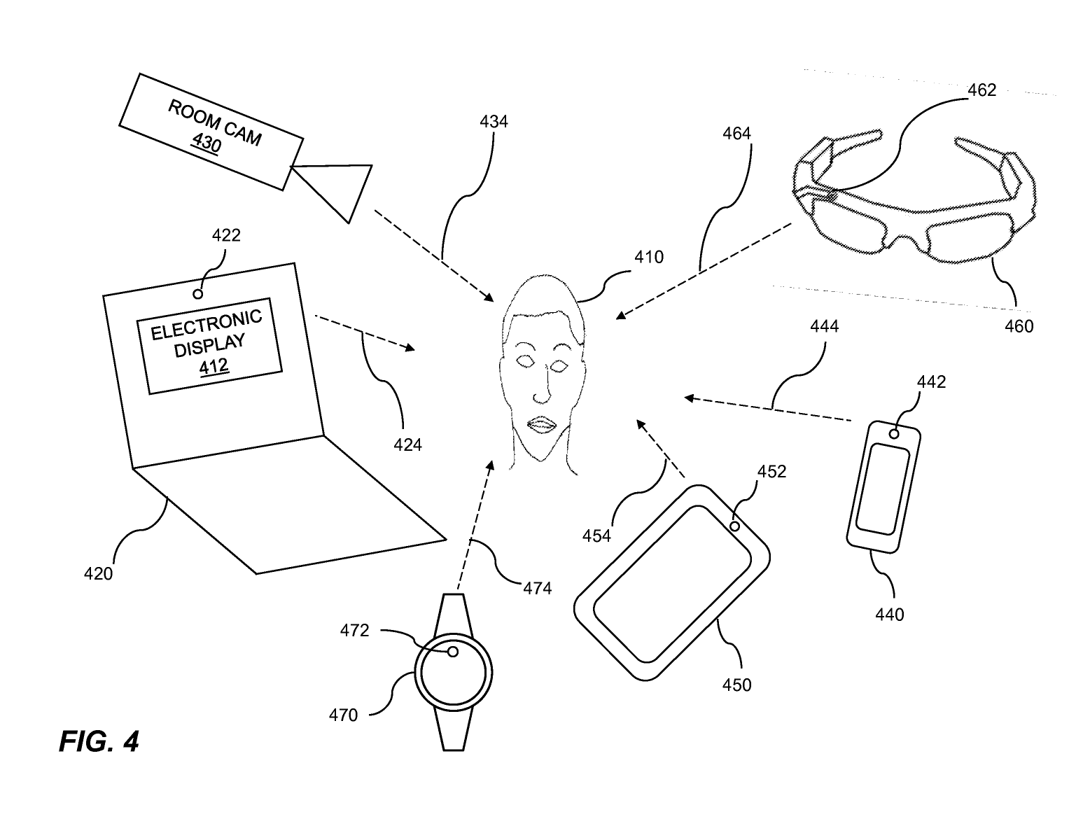

FIG. 4 is an example diagram showing collection of mental state data from multiple devices. One or more of the smart devices within FIG. 4 can have an API for emotionally enabling the device. Facial data can be collected from a plurality of sources and used for mental state analysis. In some cases only one source can be used while in other cases multiple sources can be used. A user 410 can be performing a task, viewing a media presentation on an electronic display 412, or doing any activity where it can be useful to determine the user's mental state. The electronic display 412 can be on a laptop computer 420 as shown, a tablet computer 450, a cell phone 440, a desktop computer monitor, a television, or any other type of electronic device. The mental state data can be collected on a mobile device such as a cell phone 440, a tablet computer 450, or a laptop computer 420; a fixed device, such as the room camera 430; or a wearable device such as glasses 460 or a watch 470. Thus, device can be a wearable device. Furthermore, the device can include an automobile, truck, motorcycle, or other vehicle and the mental state data can be collected by the vehicle or an apparatus mounted on or attached to the vehicle. The plurality of sources can include at least one mobile device such as a phone 440 or a tablet 450, or a wearable device such as glasses 460 or a watch 470. A mobile device can include a forward facing camera and/or rear facing camera which can be used to collect video and/or image data.

As the user 410 is monitored, the user 410 can move due to the nature of the task, boredom, distractions, or for another reason. As the user moves, the user's face can be visible from one or more of the multiple sources. For example, if the user 410 is looking in a first direction, the line of sight 424 from the webcam 422 might be able to observe the individual's face, but if the user is looking in a second direction, the line of sight 434 from the room camera 430 might be able to observe the individual's face. Further, if the user is looking in a third direction, the line of sight 444 from the phone camera 442 might be able to observe the individual's face. If the user is looking in a fourth direction, the line of sight 454 from the tablet cam 452 might be able to observe the individual's face. If the user is looking in a fifth direction, the line of sight 464 from the wearable camera 462 might be able to observe the individual's face. If the user is looking in a sixth direction, the line of sight 474 from the wearable camera 472 might be able to observe the individual's face. Another user or an observer can wear the wearable device, such as the glasses 460 or the watch 470. In other embodiments, the wearable device is a device other than glasses, such as an earpiece with a camera, a helmet or hat with a camera, a clip-on camera attached to clothing, or any other type of wearable device with a camera or other sensor for collecting mental state data. The individual 410 can also wear a wearable device including a camera which can be used for gathering contextual information and/or collecting mental state data on other users. Because the individual 410 might move his or her head, the facial data can be collected intermittently when the individual is looking in a direction of a camera. In some cases, multiple people are included in the view from one or more cameras, and some embodiments include filtering out faces of one or more other people to determine whether the individual 410 is looking toward a camera.

In some embodiments, one of the devices can run an app that can use mental state analysis. An API can be running on the device for returning mental state analysis based on the images or video collected. In some cases multiple devices can have APIs running on them for mental state analysis. In this situation the resulting analysis can be aggregated from the multiple devices after the APIs analyze the image information. In other situations, images from multiple devices are collected on one of the devices. The images are then processed through the API running on that device to yield mental state analysis.

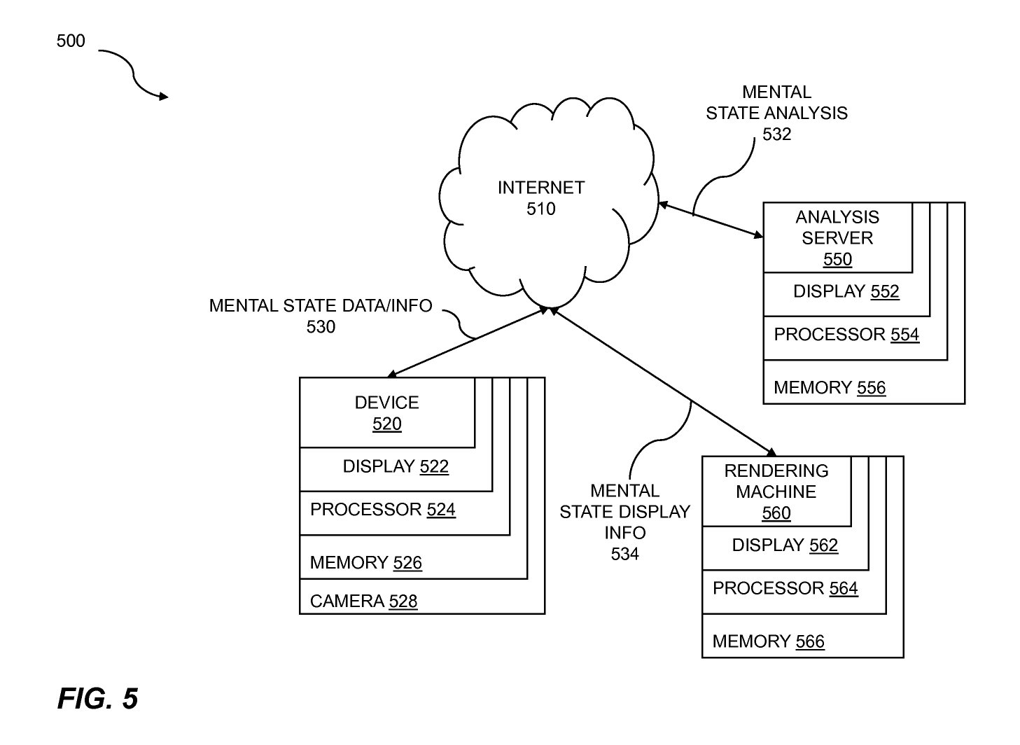

FIG. 5 is an example system for using an API for mental state analysis. The diagram illustrates an example system 500 for mental state collection, analysis, and rendering. The system 500 can include one or more client machines or devices 520 linked to an analysis server 550 via the Internet 510 or other computer network. A device 520 can include an API or SDK which provides for mental state analysis by apps on the device 520. The API can provide for mood evaluation for users of the device 520. An app can provide images to the API and the API can return mental state analysis. In the example 500 shown, the client device 520 comprises one or more processors 524 coupled to a memory 526 which can store and retrieve instructions, a display 522, and a camera 528. The memory 526 can be used for storing instructions, mental state data, mental state information, mental state analysis, and sales information. The display 522 can be any electronic display, including but not limited to, a computer display, a laptop screen, a net-book screen, a tablet computer screen, a cell phone display, a mobile device display, a remote with a display, a television, a projector, or the like. The camera 528 can comprise a video camera, still camera, thermal imager, CCD device, phone camera, three-dimensional camera, depth camera, multiple webcams used to show different views of a person, or any other type of image capture apparatus that can allow captured data to be used in an electronic system. The processors 524 of the client device 520 are, in some embodiments, configured to receive mental state data collected from a plurality of people to analyze the mental state data in order to produce mental state information. In some cases, mental state information can be output in real time (or near real time), based on mental state data captured using the camera 528. In other embodiments, the processors 524 of the client device 520 are configured to receive mental state data from one or more people, analyze the mental state data to produce mental state information and send the mental state information 530 to the analysis server 550.

The analysis server 550 can comprise one or more processors 554 coupled to a memory 556, which can store and retrieve instructions, and a display 552. The analysis server 550 can receive mental state data and analyze the mental state data to produce mental state information so that the analyzing of the mental state data can be performed by a web service. The analysis server 550 can use mental state data or mental state information received from the client device 520. The received data and other data and information related to mental states and analysis of the mental state data can be considered mental state analysis information 532. In some embodiments, the analysis server 550 receives mental state data and/or mental state information from a plurality of client machines and aggregates the mental state information.

In some embodiments, a rendering display of mental state analysis can occur on a different computer than the client device 520 or the analysis server 550. The different computer can be a rendering machine 560 which can receive mental state data 530, mental state analysis information, mental state information, and graphical display information collectively referred to as mental state display information 534. In embodiments, the rendering machine 560 comprises one or more processors 564 coupled to a memory 566, which can store and retrieve instructions, and a display 562. The rendering can be any visual, auditory, or other form of communication to one or more individuals. The rendering can include an email, a text message, a tone, an electrical pulse, or the like. The system 500 can include computer program product embodied in a non-transitory computer readable medium for mental state analysis comprising: code for obtaining images of an individual through a software interface on a device, code for evaluating the images to determine mental state data, code for analyzing mental state information based on the mental state data, and code for outputting the mental state information through the software interface. The computer program product can be part of an API that resides on a mobile device. The API can be accessed by an app running on a device to provide emotional enablement for the device.

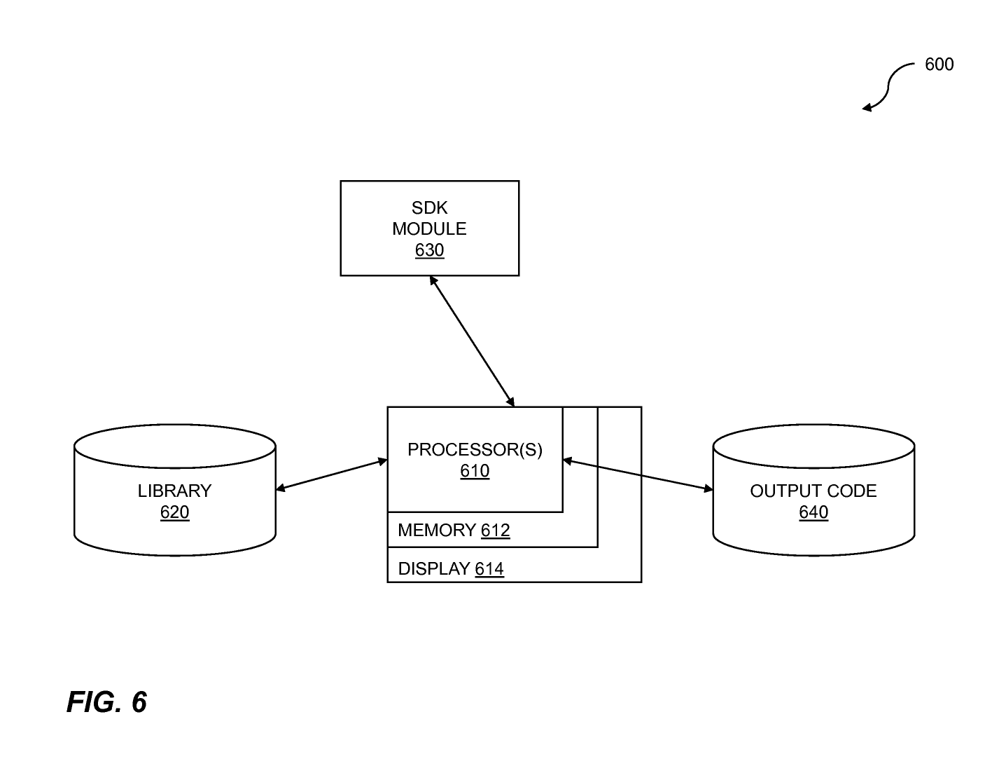

FIG. 6 is a system diagram for API generation. The system 600 can include one or more processors 610 coupled to memory 612 to store instructions and or data. A display 614 can be included in some embodiments to show results to a user. The display 614 can be any electronic display, including but not limited to, a computer display, a laptop screen, a net-book screen, a tablet computer screen, a cell phone display, a mobile device display, a remote with a display, a television, a projector, or the like. The system can include a non-transitory computer readable medium, such as a disk drive, flash memory, or another medium which can store code useful in API generation. The one or more processors 610 can access a library 620 that includes code or other information useful in API generation. In some embodiments, the library 620 can be specific for a certain type device or operating system. The processors 610 can access a software development kit (SDK) 630 which can be useful for generating an API or other code which is output 640. The API can provide for emotional enablement of devices on which the API is stored. The API can be accessed by one or more apps where the apps provide data for analysis and the API, in turn, can provide mental state analysis. The system 600 can include a computer program product embodied in a non-transitory computer readable medium for generation of APIs. In at least one embodiment the SDK module function is accomplished by the one or more processors 610.

Each of the above methods may be executed on one or more processors on one or more computer systems. Embodiments may include various forms of distributed computing, client/server computing, and cloud based computing. Further, it will be understood that for each flowchart in this disclosure, the depicted steps or boxes are provided for purposes of illustration and explanation only. The steps may be modified, omitted, or re-ordered and other steps may be added without departing from the scope of this disclosure. Further, each step may contain one or more sub-steps. While the foregoing drawings and description set forth functional aspects of the disclosed systems, no particular arrangement of software and/or hardware for implementing these functional aspects should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. Each element of the block diagrams and flowchart illustrations, as well as each respective combination of elements in the block diagrams and flowchart illustrations, illustrates a function, step or group of steps of the methods, apparatus, systems, computer program products and/or computer-implemented methods. Any and all such functions may be implemented by computer program instructions, by special-purpose hardware-based computer systems, by combinations of special purpose hardware and computer instructions, by combinations of general purpose hardware and computer instructions, by a computer system, and so on. Any and all of which implementations may be generally referred to herein as a “circuit,” “module,” or “system.”

A programmable apparatus that executes any of the above mentioned computer program products or computer implemented methods may include one or more processors, microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are not limited to applications involving conventional computer programs or programmable apparatus that run them. It is contemplated, for example, that embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized. The computer readable medium may be a non-transitory computer readable medium for storage. A computer readable storage medium may be electronic, magnetic, optical, electromagnetic, infrared, semiconductor, or any suitable combination of the foregoing. Further computer readable storage medium examples may include an electrical connection having one or more wires, a portable computer diskette, a hard disk, a random access memory (RAM), a read-only memory (ROM), an erasable programmable read-only memory (EPROM), Flash, MRAM, FeRAM, phase change memory, an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed more or less simultaneously to enhance utilization of the processor and to facilitate substantially simultaneous functions. By way of implementation, any and all methods, program codes, program instructions, and the like described herein may be implemented in one or more thread. Each thread may spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the entity causing the step to be performed.

While the invention has been disclosed in connection with preferred embodiments shown and described in detail, various modifications and improvements thereon will become apparent to those skilled in the art. Accordingly, the spirit and scope of the present invention is not to be limited by the foregoing examples, but is to be understood in the broadest sense allowable by law.

What is claimed is:

1. A computer-implemented method for application programming interface usage comprising:
obtaining images of an individual through a software interface on a device;
evaluating the images to determine mental state data;
analyzing mental state information based on the mental state data; and
outputting the mental state information through the software interface.

2. The method of claim 1 further comprising receiving a connection request through the software interface for the images and the mental state information.

3. The method of claim 2 wherein the connection request comes from a peer component.

4. The method of claim 3 wherein the peer component is also running on the same device as the application programming interface.

5. The method of claim 2 further comprising receiving a connection data structure for the connection request where the connection data structure describes a format for the images.

6. The method of claim 2 further comprising receiving a connection data structure for the connection request where the connection data structure describes a format for the mental state information.

7. (canceled)

8. The method of claim 1 wherein the software interface includes an application programming interface (API).

9. The method of claim 8 wherein the application programming interface is generated by a software development kit (SDK).

10. (canceled)

11. The method of claim 9 wherein the software development kit runs on a different system from the device.

12. The method of claim 1 further comprising delegating a message from the software interface to an app.

13-15. (canceled)

16. The method of claim 1 wherein the analyzing includes inferring mental states based on the mental state data.

17. The method of claim 16 wherein the mental states include one or more of stress, sadness, anger, happiness, disgust, frustration, confusion, disappointment, hesitation, cognitive overload, focusing, engagement, attention, boredom, exploration, confidence, trust, delight, disgust, skepticism, doubt, satisfaction, excitement, laughter, calmness, or curiosity.

18. The method of claim 1 wherein the analyzing is based on one or more classifiers.

19. The method of claim 18 further comprising downloading the one or more classifiers from a web service.

20. The method of claim 19 wherein the one or more classifiers for downloading are based on one or more of processing capability of the device, bandwidth capability for the device, memory amount on the device.

21. The method of claim 19 further comprising updating the one or more classifiers.

22. The method of claim 21 wherein the updating includes tuning the one or more classifiers based on improvements to the one or more classifiers in cloud storage.

23. The method of claim 1 wherein the mental state information, which is output, is used to determine smiles, project sales, determine expressiveness, analyze media, monitor wellbeing, evaluate blink rate, evaluate heart rate, evaluate humor, or is posted to a social network.

24. The method of claim 1 further comprising assembling mental state data from multiple devices and using that mental state data in the analyzing.

25. The method of claim 1 wherein the mental state data is collected sporadically.

26. The method of claim 1 further comprising obtaining images for a plurality of people; evaluating the images for the plurality of people to determine mental state data; analyzing mental state information for the plurality of people based on the mental state data for the plurality of people; and outputting the mental state information for the plurality of people through the software interface.

27. The method of claim 1 wherein the images are retrieved from storage.

28. (canceled)

29. The method of claim 27 wherein the storage is across a network to which the device is coupled.

30. (canceled)

31. The method of claim 1 wherein the images are received from an active webcam.

32. The method of claim 1 wherein the images contain a plurality of faces and where mental state information is analyzed for multiple people associated with the plurality of faces.

33. (canceled)

34. A computer program product embodied in a non-transitory computer readable medium for mental state analysis, the computer program product comprising:
code for obtaining images of an individual through a software interface on a device;
code for evaluating the images to determine mental state data;
code for analyzing mental state information based on the mental state data; and
code for outputting the mental state information through the software interface.

35. A computer system for mental state analysis comprising:
a memory which stores instructions;
one or more processors attached to the memory wherein the one or more processors, when executing the instructions which are stored, are configured to:
obtain images of an individual through a software interface on a device;
evaluate the images to determine mental state data;
analyze mental state information based on the mental state data; and
output the mental state information through the software interface.
